(19) United States
(12) Patent Application Publication
(10) Pub. No.: US 2006/0194327 A1
(43) Pub. Date: Aug. 31, 2006

(54) On-chip magnetic particle sensor with improved snr

(75) Inventors: Josephus Arnoldus Henricus Maria Kahlan, Eindhoven (NL); Menno Willem Jose Prins, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 10/566,556

(22) PCT Filed: Jul. 27, 2004
(86) PCT No.: PCT/IB04/51297

§ 371 Date: Jan. 27, 2006

(30) Foreign Application Priority Data
Jul. 30, 2003 (EP) 03102353.4

(51) Int. Cl. G01N 33/00; G01V 3/00; A61B 5/05

(52) U.S. Cl. 436/86; 600/412; 324/313

(57) ABSTRACT

A device and method is disclosed for the detection or determination of the presence of magnetic particles (15), such as for example, but not limited to, magnetic tianoparticles. In particular it relates to an integrated or on-chip magnetic sensor element (11) for the detection of magnetic particles. The device and method of the present invention o er high signal-to-noise ratio and low power consumption and do not require an external magnetic field. They may be used for magnetic detection of binding of biological molecules on a micro-array or biochip.

The invention relates to a magnetic sensor device for determining the presence of at least one magnetic particle, the magnetic sensor device comprising:

a magnetic sensor element on a substrate,

a magnetic field generator for generating an ac magnetic field,

a sensor circuit comprising the magnetic sensor element for sensing a magnetic property of the at least one magnetic particle which magnetic property is related to the ac magnetic field.

The invention further relates to a method for determining the presence of at least one magnetic particle, the method comprising the steps of

generating an ac magnetic field in the vicinity of a magnetic sensor element,

sensing with the magnetic sensor element a magnetic property of the at least one magnetic particle which magnetic property is related to the ac magnetic field.

The introduction of micro-arrays or biochips is revolutionizing the analysis of samples for DNA (desoxyribonucleic acid), RNA (ribonucleic acid), proteins, cells and cell fragments, tissue elements, etc. Applications are e.g. human genotyping (e.g. in hospitals or by individual doctors or nurses), bacteriological screening, biological and pharmacological research.

Biochips, also called biosensor chips, biological microchips, gene-chips or DNA chips, consist in their simplest form of a substrate on which a large number of different probe molecules are attached, on well defined regions on the chip, to which molecules or molecule fragments that are to be analyzed can bind if they are perfectly matched. For example, a fragment of a DNA molecule binds to one unique complementary DNA (c-DNA) molecular fragment. The occurrence of a binding reaction can be detected, e.g. by using fluorescent markers that are coupled to the molecules to be analyzed. This provides the ability to analyze small amounts of a large number of different molecules or molecular fragments in parallel, in a short time. One biochip can hold assays for 10-1000 or more different molecular fragments. It is expected that the usefulness of information that can become available from the use of biochips will increase rapidly during the coming decade, as a result of projects such as the Human Genome Project, and follow-up studies on the functions of genes and proteins.

G. Li et al. describe in “Detection of single micron-sized magnetic bead and magnetic nanoparticles using spin valve sensors for biological applications”, Journal of Applied Physics, Vol. 93, number 10, pp. 7557-7559, 15 May 2003, a series of spin-valve sensors for the detection of a single superparamagnetic bead. The magnetic beads are labels for biological molecules.

The sensor chip comprises a Wheatstone bridge configuration with a pair of sensor (Rsen) and reference strips (Rref) on the chip and two off-chip resistors (R1 and R2). The sensor chip is placed in a gap of two orthogonal electromagnets in such a way that the longitudinal direction of the spin valve strips is aligned with a dc bias field Hb and the transverse direction parallel to an ac tickling field Ht.

By polarizing the magnetic microbead on the spin valve sensor with the dc magnetic field and modulating its magnetization with the orthogonal ac magnetic field, one observed a magnetoresistance (MR) signal reduction caused by the magnetic dipole field from the bead that partially cancelled the applied fields to the spin valve. A lock-in technique was used to measure a voltage signal due to the MR reduction.

When the beads were removed, a jump in the signal well above the noise level was observed, indicating the difference between the initial state (presence of a single bead) and the detection state (absence of the bead).

It is a disadvantage of the above system that the achievable signal-to-noise ratio (SNR) is limited. For instance, the sensor in the Wheatstonebridge configuration has a Reference strip (Rref) of magnetoresistive material that introduces additional unwanted noise. Due to the high noise level, the system is not capable to detect the signal of a single bead, only the difference between the presence or absence of a single bead.

It is an object of the present invention to provide a device of the type mentioned in the opening paragraph, the device having an improved signal to noise ratio (SNR).

The object according to the invention is achieved in that the magnetic field generator is present on the substrate and is arranged to operate at a frequency of 100 Hz or above.

The noise level of the magnetic sensor device is determined by several noise sources such as by the presence of (magnetic) 1/f noise in the magnetic sensor elements itself, by the electronic noise properties of the electronic sensing circuit such as amplifiers used (e.g. noise, offset, drift) and by unwanted magnetic fields. The invention is based on the insight that in the low frequency regime, at frequencies e.g. below 100 Hz, the 1/f noise of the magnetic sensor element dominates. 1/f noise is caused by point-to-point fluctuations of the current and is proportional to the inverse of the frequency. In magnetoresistive sensors 1/f noise originates from magnetic fluctuations in the free layer. When the frequency of the generated ac magnetic field is 100 Hz or above, the dominating 1/f noise is significantly reduced compared to the prior art (e.g. Li uses 40 Hz), resulting in an improved signal to noise ratio (SNR).

It is advantageous when the frequency of the ac magnetic field is further increased to a value where the thermal white (Nyquist) noise level becomes dominant over the 1/f noise level. To the surprise of the inventors it turned out that in GMR sensors above a certain corner frequency f_{c}≈50 kHz the thermal white noise becomes dominant. The white-noise level limits the theoretically achievable detection limit.

In order to be able to generate an ac magnetic field with a high frequency, a conductor integrated on the substrate is used through which an ac current is sent. The frequency of the alternating magnetic field can be much higher than in the prior art, where electromagnets are used. These electromagnets can only operate at low frequencies of about 1-40 Hz. An additional advantage of using a conductor such as a wire, a strip etc, is that relatively low power is needed compared to the electromagnet of the prior art. A further advantage is that the magnetic field generator is mechanically aligned to the magnetic sensing layer in a well-defined way. This avoids the need for careful alignment between electromagnet and sensor during a measurement procedure.

The magnetic field generator and the sensing circuit can be integrated on one chip. This allows a very compact system. Moreover when a plurality of magnetic sensor elements are present for the detection of magnetic particles functioning as labels to biological molecules on an array or biochip, integration of all the connections to the sensor elements and the sensing circuits becomes much easier on chip than off chip. Thin film technologies allows multilevel metallization schemes and compact integrated circuit design.

The substrate can contain electronics that fulfill all detection and control functions (e.g. locally measurement of temperature and pH). This has the following advantages:

it makes the use of expensive and large (optical) detection systems unnecessary,

it provides the possibility to further enhance the areal density of probed molecules,

it enhances speed, accuracy and reliability,

it decreases the amount of test volume required, and

it decreases labor cost.

Biochips can become a mass product when they provide an absolutely inexpensive method for diagnostics, regardless of the venue (not only in hospitals but also at the sites of individual doctors), and when their use leads to a reduction of the overall cost of disease management.

Magnetoresistive sensors based on GMR and TMR elements can advantageously be used to measure slowly varying processes such as in the field of molecular diagnostics (MDx). Using magnetoresistive materials, a rugged, single-component, micro-fabricated detector may be produced, that will simultaneously monitor tens, hundreds, thousands or even millions of experiments.

In an advantageous embodiment the magnetic sensor element lies in a plane and there is a plurality of magnetic generators present.

The plurality of magnetic field generators can be located at different levels with respect to the plane of the magnetic sensor element.

It is a further object of the present invention to provide a method of the type mentioned in the opening paragraph, the method for detection of magnetic particles resulting in an improved signal to noise ratio (SNR).

The object according to the invention is achieved in that the frequency of the ac magnetic field is chosen at 100 Hz or above.

Preferably the frequency is chosen at a value where the thermal white (Nyquist) noise of the magnetic sensor element dominates the 1/f noise of the magnetic sensor element. The noise level in the detection system is dominated by the noise spectrum of the magnetic sensor element The magnetic sensor element can be a GMR or TMR sensor. In those sensors based on the magnetoresistance effect, the 1/f noise is caused by fluctuations of the magnetization direction of the free layer of the sensor. The free layer is the sensitive layer in the GMR or TMR sensor.

When there is a plurality of magnetic generators present, the method can be used advantageously for determining a concentration of magnetic particles as a function of location of the magnetic particles, e.g. in a biological sample such a micro-array or biochip.

When the plurality of magnetic field generators are located at different levels with respect to the plane of the magnetic sensor element, the method allows the distinction and determination of the surface concentration and the bulk concentration of the magnetic particles. Further, the method is suitable to determine the position of the magnetic particles in a direction perpendicular to the plane of the magnetic sensor element, as well as the position parallel to a plane of the magnetic sensor element.

For accurate measurements, a calibration method can be applied. First the magnetic field generated by the magnetic field generator(s) is measured in absence of magnetic particles. The measurement value is subtracted from the actual measurement value obtained when a measurement is carried out in the presence of magnetic particles.

The calibrating measurement value can be stored in a memory, such as an MRAM, which can be electronically integrated with the magnetic sensor element and the sensing circuit on one chip.

Because there is no need for application of an off-chip generated external magnetic field, the noise level can further be reduced, and thus enables more accurate measurements. A further advantage is the smaller form factor of the (bio)sensor interface configuration.

These and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference Figs. quoted below refer to the attached drawings.

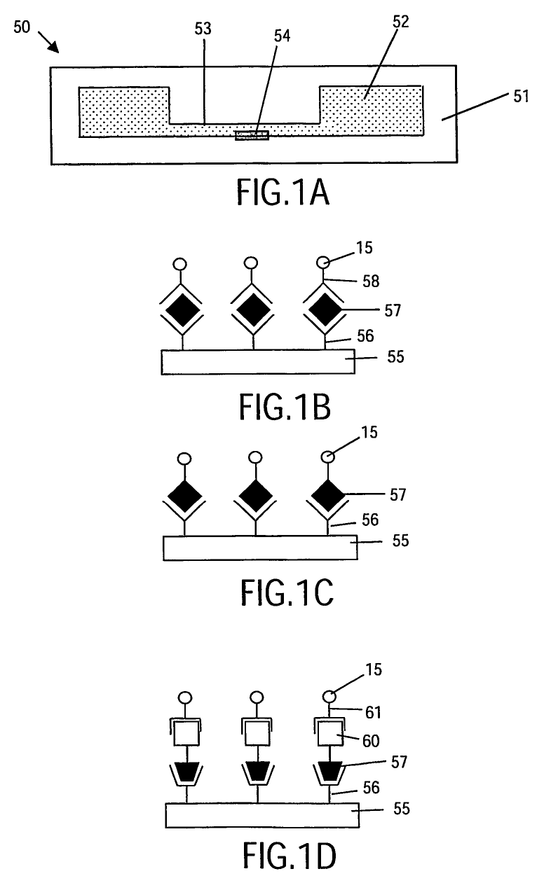

FIG. 1A shows a schematic representation of a biosensor device.

FIGS. 1B, 1C and 1D show details of a probe element provided with binding sites able to selectively bind target sample, and magnetic nanoparticles being directly or indirectly bound to the target sample in different ways.

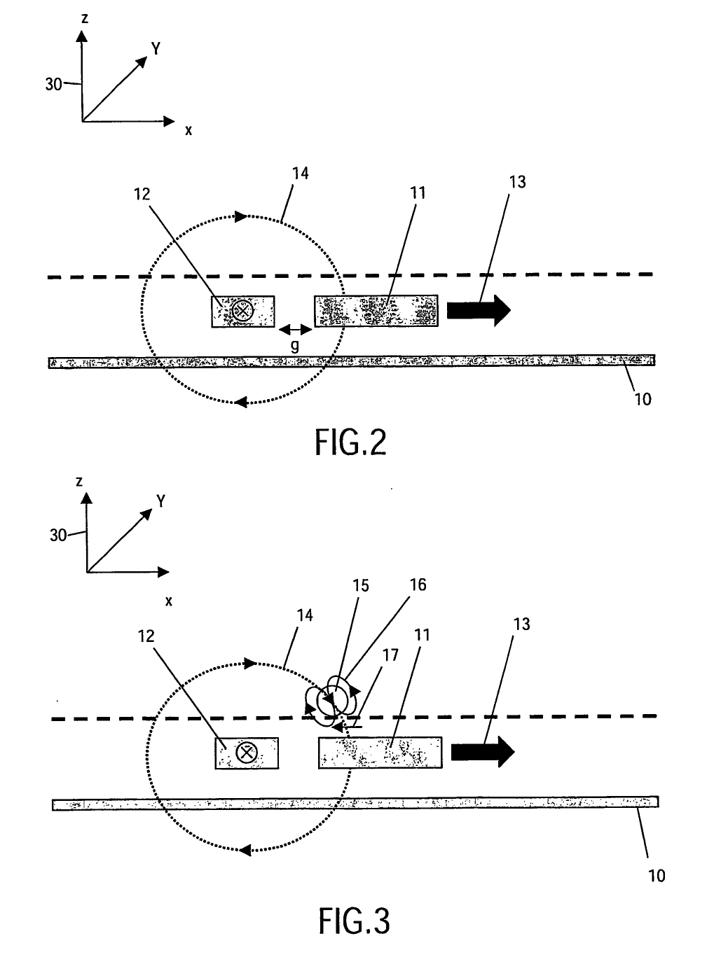

FIG. 2 is a cross-sectional view of a sensor device according to a first embodiment of the present invention in absence of magnetic particles.

FIG. 3 is a cross-sectional view of a sensor device according to the first embodiment of the present invention in the presence of magnetic particles.

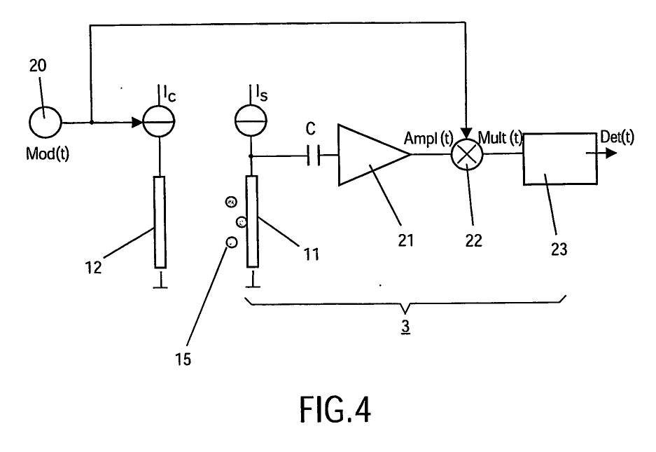

FIG. 4 is a schematic view of a detection method according to the first embodiment of the present invention.

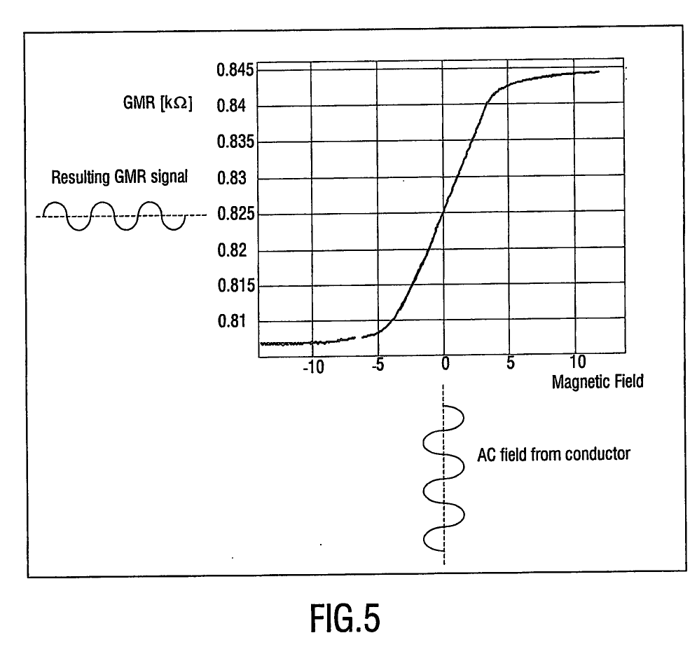

FIG. 5 shows the magnetoresistance characteristic of a GMR sensor element, the ac magnetic field, and the resulting GMR output signal.

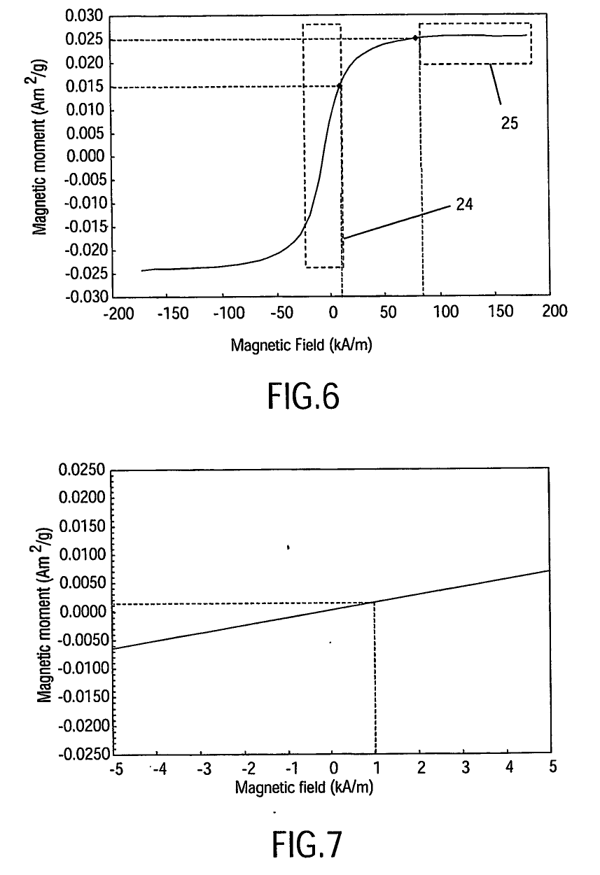

FIG. 6 is a graph of the magnetic moment of a magnetic nano-particle as a function of an applied magnetic field.

FIG. 7 is a detail of the magnetization curve of FIG. 6.

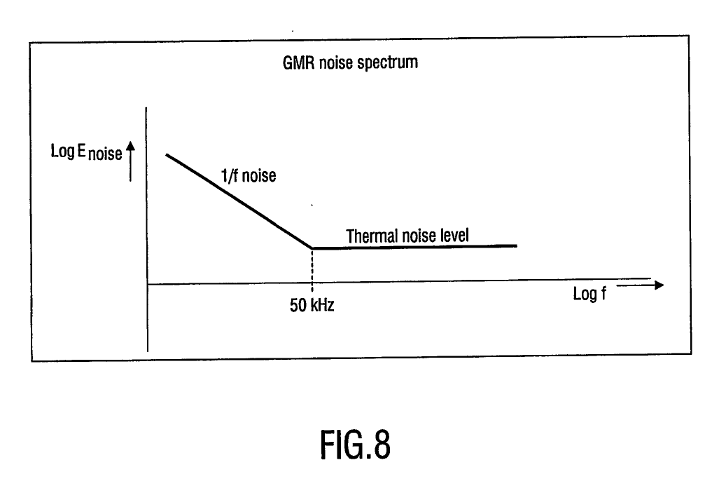

FIG. 8 shows schematically the dominant noise spectrum of the GMR sensor element.

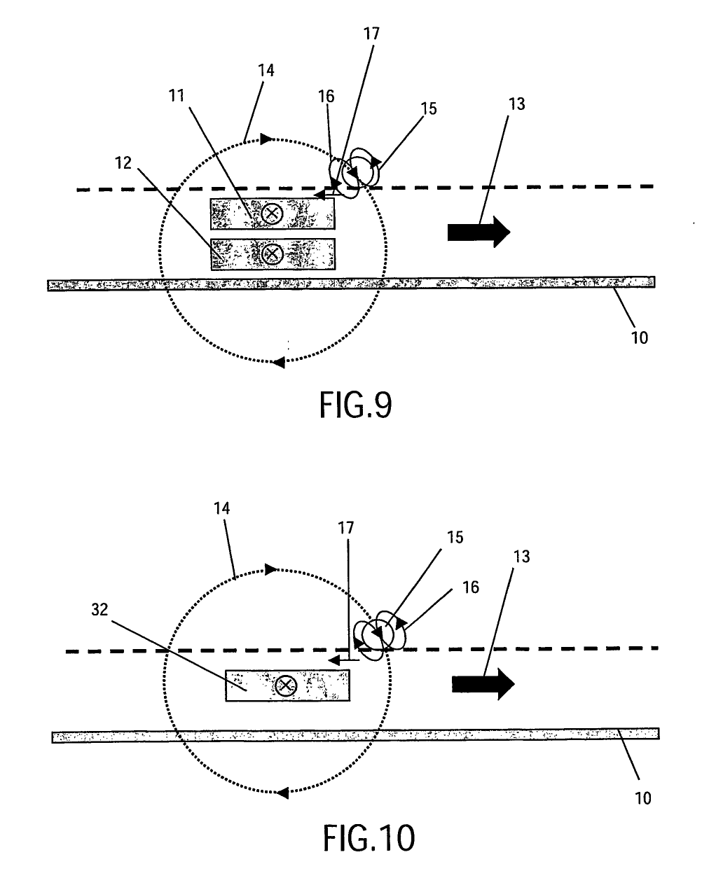

FIG. 9 is a cross-sectional view of a sensor device according to a second embodiment of the present invention.

FIG. 10 is a cross-sectional view of a sensor device according to a third embodiment of the present invention.

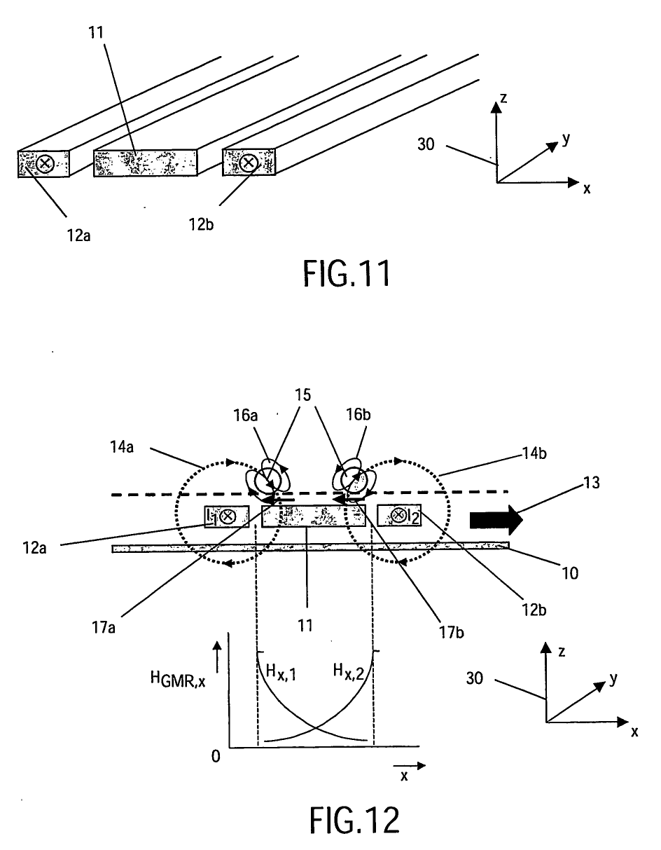

FIG. 11 shows a combination of a magnetic sensor with two conductors as used in an fourth embodiment of the present invention.

FIG. 12 is a cross sectional view of a sensor device according to the fourth embodiment of the present invention.

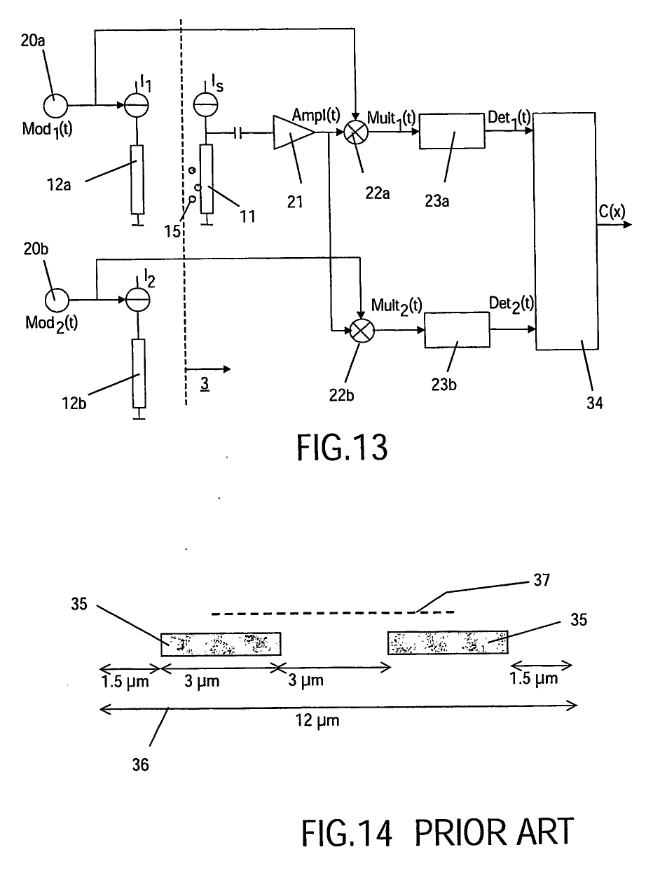

FIG. 13 is a schematic view of a detection method for use with the sensor device according to the fourth embodiment of the present invention.

FIG. 14 is a cross section of a sensor described in the prior art and illustrating chip area dimensions.

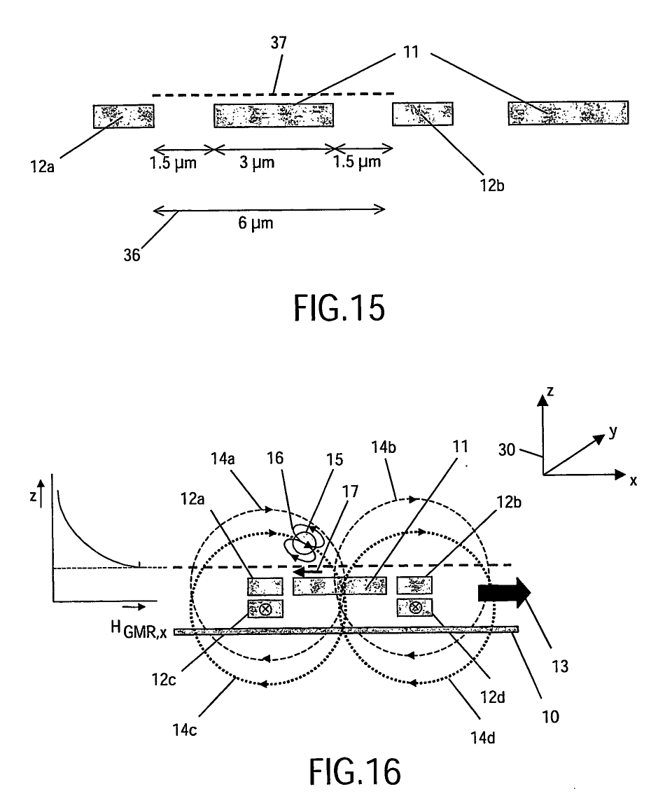

FIG. 15 is a cross section of a sensor device according to the fourth embodiment of the present invention showing chip area dimensions.

FIG. 16 is a cross sectional view of a sensor device according to a fifth embodiment of the present invention.

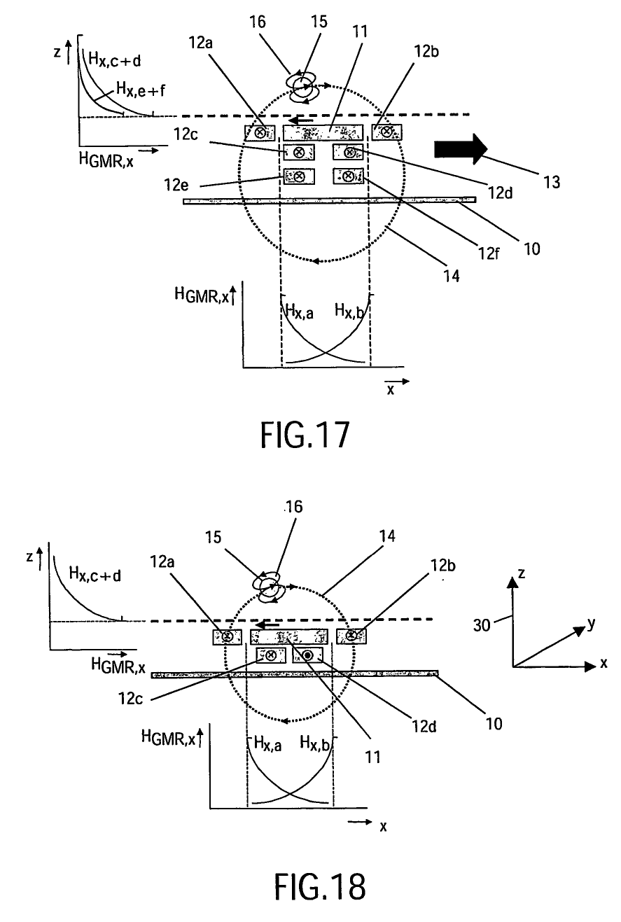

FIG. 17 is a cross sectional view of a sensor device according to a sixth embodiment of the present invention.

FIG. 18 is a cross sectional view of a sensor device according to an seventh embodiment of the present invention.

In the different Figs., the same reference Figs. refer to the same or analogous elements.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

A biosensor device 50 is represented schematically in FIG. 1A. It comprises a cartridge housing 51, chambers 52 and/or channels for containing the material, e.g. analyte to be analyzed, and a biochip 54. The biochip 54 is a collection of miniaturized test sites (micro-arrays) arranged on a solid substrate that permits many tests to be performed at the same time in order to achieve higher throughput and speed. It can be divided into tens to thousands of tiny chambers each containing bioactive molecules, e.g. -short DNA strands or probes. It can be three dimensional, capable of running as many as 10,000 different assays at the same time. Or, the chip 54 can be manufactured more simply with as few as 10 different assays running at one time. In addition to genetic applications (decoding genes), the biochip 54 is being used in toxicological, protein, and biochemical research, in clinical diagnostics and scientific research to improve disease detection, diagnosis and ultimately prevention A biochip 54 comprises a substrate with at its surface at least one, preferably a plurality of probe areas. Each probe area comprises a probe element 55 over at least part of its surface. The probe element 55 is provided with binding sites 56, such as for example binding molecules or antibodies, able to selectively bind a target sample 57 such as for example a target molecule species or an antigen. Any biologically active molecule that can be coupled to a matrix is of potential use in this application. Examples are:

Nucleic acids: DNA, RNA double or single stranded or DNA-RNA hybrids, with or without modifications. Nucleic acid arrays are well known.

Proteins or peptides, with or without modifications, e.g. antibodies, DNA or RNA binding proteins. Recently, grids with the complete proteome of yeast have been published.

Oligo- or polysaccharides or sugars

Small molecules, such as inhibitors, ligands, cross-linked as such to a matrix or via a spacer molecule.

The items spotted on the grid will be most likely libraries of compounds, such as peptide/protein libraries, oligonucleotides libraries, inhibitor libraries.

There exist different possibilities to connect magnetic particles to a target sample, examples of which are shown in FIGS. 1B, 1C and 1D. Different types of magnetic particles which can be used with the present invention are described by Urs Häfeli et al. in “Scientific and Clinical Applications of Magnetic Carriers”, Plenum Press, New York, 1597, ISBN 0-306-45687-7.

In FIG. 1B, sensor molecules 58 labeled with magnetic particles 15 are able to selectively bind target sample 57. When random searches are performed, e.g. screening in which DNA binding proteins of a certain tissue extract bind to a grid with a library of nucleotides, the sensor molecule should have a very broad specificity. In this example a sensor molecule with a spacer reactive towards amino groups or carboxy groups would be useful. Other sensor molecules with a reactive group towards sugars, DNA are also suitable. In the case of a direct search, tailor-made sensor molecules can be used e.g. where a screening with a protein against a protein library is performed for assumed protein-protein interaction, an antibody is an obvious choice. Both monoclonal and polyclonal antibodies may be used. As shown in FIG. 1B, magnetic particles 15 are indirectly bound to the target sample 57.

In FIG. 1C, the target sample 57 molecules are directly labeled by magnetic particles 15.

In FIG. 1D, target sample 57 is labeled by labels 60. Such a labeled target sample 57 (e.g. biotinylated sample DNA) is selectively bound to binding sites 56. Sensor molecules 61 (e.g. streptavidin) labeled with magnetic particles 15 are able to selectively bind the labels 60 on the target sample 57. Again, the magnetic particles 15 are indirectly bound to the target sample 57.

The functioning of the biochip 54 is as follows. Each probe element 55 is provided with binding sites 56 of a certain type. Target sample 57 is presented to or passed over the probe element 55, and if the binding sites 56 and the target sample 57 match, they bind to each other. Magnetic particles 15 are directly or indirectly coupled to the target sample 57, as illustrated in FIGS. 1B, 1C and 1D. The magnetic particles 15 allow to read out the information gathered by the biochip 54.

The present invention is about how to read out the information gathered by the biochip 54 by means of a magnetic sensor device. In the following the present invention will be described referring to magnetoresistive devices, such as AMR, GMR or TMR devices, as part of the magnetic sensor device. However, the invention is not limited thereto and can make use of any suitable kind of magnetic sensor element, such as for example a Hall sensor or a SQUID (superconducting quantum interference device).

In a first embodiment the device according to the present invention is a biosensor and will be described with respect to FIG. 2 and FIG. 3. The biosensor detects magnetic particles in a sample such as a fluid, a liquid, a gas, a visco-elastic medium, a gel or a tissue sample. The magnetic particles can have small dimensions. With nanoparticles are meant particles having at least one dimension ranging between 0.1 nm and 1000 nm, preferably between 3 nm and 500 nm, more preferred between 10 nm and 300 nm. The magnetic particles can acquire a magnetic moment due to an applied magnetic field (e.g. they can be paramagnetic) or they can have a permanent magnetic moment. The magnetic particles can be a composite, e.g. consist of one or more small magnetic particles inside or attached to a non-magnetic material. As long as the particles generate a non-zero response to the frequency of an ac magnetic field, i.e. when they generate a magnetic susceptibility or permeability, they can be used.

The device may comprise a substrate 10 and a circuit e.g. an integrated circuit. A measurement surface of the device is represented by the dotted line in FIG. 2 and FIG. 3. In embodiments of the present invention, the term “substrate” may include any underlying material or materials that may be used, or upon which a device, a circuit or an epitaxial layer may be formed. In other alternative embodiments, this “substrate” may include a semiconductor substrate such as e.g. a doped silicon, a gallium arsenide (GaAs), a gallium arsenide phosphide (GaAsP), an indium phosphide (InP), a germanium (Ge), or a silicon germanium (SiGe) substrate. The “substrate” may include for example, an insulating layer such as a SiO₂ or an Si₃N₄ layer in addition to a semiconductor substrate portion. Thus, the term substrate also includes glass, plastic, ceramic, silicon-on-glass, silicon-on sapphire substrates. The term “substrate” is thus used to define generally the elements for layers that underlie a layer or portions of interest Also, the “substrate” may be any other base on which a layer is formed, for example a glass or metal layer. In the following reference will be made to silicon processing as silicon semiconductors are commonly used, but the skilled person will appreciate that the present invention may be implemented based on other semiconductor material systems and that the skilled person can select suitable materials as equivalents of the dielectric and conductive materials described below.

The circuit may comprise a magnetoresistive sensor 11 as a sensor element and a magnetic field generator in the form of a conductor 12. The magnetoresistive sensor 11 may for example be a GMR or a TMR type sensor. The magnetoresistive sensor 11 may for example have an elongated, e.g. a long and narrow stripe geometry but is not limited to this geometry. Sensor 11 and conductor 12 may be positioned adjacent to each other (FIG. 2) within a close distance g. The distance g between sensor 11 and conductor 12 may for example be between 1 nm and 1 mm; e.g. 3 μm. The minimum distance is determined by the IC process.

In FIGS. 2 and 3, a co-ordinate system is introduced to indicate that if the sensor device is positioned in the xy plane, the sensor 11 mainly detects the x-component of a magnetic field, i.e. the x-direction is the sensitive direction of the sensor 11. The arrow 13 in FIG. 2 and FIG. 3 indicates the sensitive x-direction of the magnetoresistive sensor 11 according to the present invention. Because the sensor 11 is hardly sensitive in a direction perpendicular to the plane of the sensor device, in the drawing the vertical direction or z-direction, a magnetic field 14, caused by a current flowing through the conductor 12, is not detected by the sensor 11 in absence of magnetic nano-particles 15. By applying a current to the conductor 12 in the absence of magnetic nano-particles 15, the sensor 11 signal may be calibrated. This calibration is preferably performed prior to any measurement.

When a magnetic material (this can e.g. be a magnetic ion, molecule, nano-particle 15, a solid material or a fluid with magnetic components) is in the neighborhood of the conductor 12, it develops a magnetic moment m indicated by the field lines 16 in FIG. 3. The magnetic moment m then generates dipolar stray fields, which have in-plane magnetic field components 17 at the location of the sensor 11. Thus, the nano-particle 15 deflects the magnetic field 14 into the sensitive x-direction of the sensor 11 indicated by arrow 13 (FIG. 3). The x-component of the magnetic field Hₓ which is in the sensitive x-direction of the sensor 11, is sensed by the sensor 11 and depends on the number Nₙₚ of magnetic nano-particles 15 and the conductor current I_{c}.

A method for detection of magnetic nano-particles, according to an embodiment of the present invention, is illustrated in FIG. 4. A modulating signal Mod(t) having a suitable waveform such as a sinusoidal wave (sin at) and with a high frequency of, for example but not limited thereto, 50 kHz, coming from a source 20, is sent to the conductor 12 to modulate the conductor current I_{c}. With a “high frequency” according to the present invention is meant a frequency which does not generate a substantial movement of the magnetic particles at that frequency, for example a frequency of 100 Hz or higher, preferably 1 kHz or higher, more preferred 50 kHz or higher.

The conductor current is modulated such that I_{c}=I_{c} sin at, and this modulated current induces a magnetic field which per se is mainly vertical or z-oriented at the location of the magnetoresistive sensor 11, as shown by the field line 14 in FIG. 2.

A sensing current Iₛ passes through the magnetoresistive sensor 11. Depending on the presence of nano-particles 15 in the neighborhood of the magnetoresistive sensor 11, the magnetic field at the location of the sensor 11, and thus the resistance of the sensor 11 is changed.

FIG. 5 shows the magnetoresistance characteristic of the GMR sensor. Without the presence of magnetic particles, the input signal is the ac magnetic field from the conductor. Depending on the presence of nano-particles 15 in the neighborhood of the magnetoresistive sensor 11, the magnetic field at the location of the sensor 11, and thus the resistance of the sensor 11 is changed. The magnetic field Hₓ in the sensitive x-direction of the magnetoresistive sensor 11 is to a first order proportional to the number Nₙₚ of magnetic nanoparticles and the conductor current I_{c}:
Hₓ∝NₙₚI_{c} sin at.

A different resistance of the sensor 11 leads to a different voltage drop over the sensor 11, and thus to a different measurement signal delivered by the sensor 11. The response to the ac magnetic field signal is shown schematically on the left hand side of FIG. 5. The resulting GMR output signal is a continuous wave.

The measurement signal delivered by the magnetoresistive sensor 11 is then delivered to an amplifier 21 for amplification thus generating an amplified signal Ampl(t).

This amplified signal Ampl(t) is detected, synchronously demodulated by passing through a demodulating multiplier 22 where the signal is multiplied with the modulation signal Mod(t) (in this case equal to sin at), resulting in an intermediate signal Mult(t), the intermediate signal Mult(t) being equal to:
Mult(t)=NₙₚI_{c} sin² at=NₙₚI_{c}.1/2(1−cos 2at).

In a last step, the intermediate signal Mult(t) is sent through a low pass filter 23. The resulting signal Det(t) is then proportional to the number Nₙₚ of magnetic nano-particles 15 present at the surface of the sensor 11.

Additionally, the amplifier 21 can be AC coupled to the magnetoresistive sensor 11 by means of a low-frequency suppressor such as a capacitor C. The capacitor further enhances the low-frequency suppression.

In the present invention, magnetic particles, e.g. magnetic nano-particles 15, are operated in their linear region 24 which means that the magnetic moment m of the magnetic particles 15 linearly follows the magnetic field strength (FIG. 6). This also means that only a small magnetic field is required to induce a magnetic moment in the nano-particles 15. For example, for nano-particles having a diameter of 50 nm, the full linear range 24 of the magnetic moment m versus the magnetic field can amount from −0.015 Am²/g to +0.015 Am²/g, requiring from −10 kA/m to +10 kA/m magnetic field strength. In case that magnetic nano-particles 15 are operated in the saturated region 25 a much higher magnetic field is required, i.e. at least 80 kA/m. From FIG. 6 the signal loss in linear versus saturated operation can be calculated and equals mₗᵢₙ/mₛₐₜ=0.015/0.025=0.6.

In the proposed embodiment, a magnetic moment is induced by a magnetic field with low field strength, which in its turn is induced by a magnetic field generator such as a current flowing in a conductor 12. If, in a specific example, the sensor 11 has an elongated, i.e. long and narrow, stripe geometry and the distance between the conductor 12 and the sensor 11 is g=3 μm, with a conductor current with an amplitude I_{c}=20 mA, the vertical field strength equals H_{z}=I/2.w≈1 kA/m. A detailed view of the magnetization curve of FIG. 6 shows that the magnetization at 1 kA/m equals 0.0015 Am²/g (FIG. 7). With respect to the saturated case, the detected signal has decreased by a factor 0.0015/0.025=0.06.

By applying the detection method as described in FIG. 4, the noise can be reduced. This will be illustrated in the following discussion.

FIG. 8 shows schematically the dominant noise source of the detection system of FIG. 4. At low frequencies of the ac magnetic field, the 1/f noise of the GMR sensor element dominates all other electronic noise sources.

Under the condition that the detection is 1/f noise limited, which is the case in this embodiment, the SNR loss may be compensated by increasing the modulation frequency from for example 10 Hz to f_{mod}=(1/0.06²).10=2.8 kHz. The SNR can be further enhanced by increasing the modulation frequency f_{mod} to the point where the thermal noise dominates, which is typically 50 kHz. This will lead to a net improvement of (50/2.8)^{1/2}=4=12 dB with respect to the method discussed in WO 03054523. By lowering the amplifier thermal noise floor level, it becomes sensible to increase the modulation frequency f_{mod} beyond 50 kHz so that the SNR will improve further.

Next to the improvement of the signal-to-noise ratio, another advantage of the detection method described in this embodiment is that no external magnetic field from outside the chip has to be provided. Sending a modulating signal through the conductor 12 creates the magnetic field.

Furthermore, the magnetic particles used do not need to be large; they may have a small magnetic moment as no movement of the magnetic particles is needed for detection. Also detection can be carried out both during application of the magnetic field or during relaxation thereof, so it is not necessary to provide large particles having a sufficiently long relaxation time.

Another advantage of this embodiment is that (bio)chemical structuring of the sensor is not needed. The (bio)chemical structuring may comprise:

(1) surface patterning. This refers to patterning of a surface, where the pattern is in some way aligned to other structures on or in a substrate. The pattern can consist of a monolayer of molecules, of a thin-film material, or even of material that has been removed.

(2) surface modification. This refers to a (bio)chemical modification of a surface, for example to couple specific capture molecules to a surface. A surface modification can be applied in a patterned fashion, e.g. aligned with respect to sensors in a substrate.

Conventional particle sensors, when applied to biosensors, have generally been provided with some kind of surface structure to be able to bind target molecules to their surface in order to determine the concentration of the target molecules in the solution to be analyzed. In the case of the present invention, this surface structure is no longer necessary or much simpler because very locally a non-uniform magnetic field is applied. A signal will be detected even when the surface is covered with a homogeneous distribution of magnetic particles.

A further advantage is the possibility to perform several measurements in parallel, instead of successively. This is due to the fact that the magnetic field of each conductor is locally concentrated, so different magnetic fields (frequency, amplitude, etc.) can be used on different spots.

In a second embodiment, a detection method described in any of the previous embodiments is applied with different device geometry. The device geometry described in this embodiment is illustrated schematically in FIG. 9. The conductor 12 is now positioned between the substrate 10 and the magnetoresistive sensor 11. In this case, in order to be able to do a measurement, a preliminary, calibrating measurement needs to be carried out in absence of magnetic particles 15, which calibrating measurement measures the magnetic field generated by the on-chip magnetic field generator 11. The obtained calibrating measurement value is then used thereafter and is subtracted from the actual measurement value obtained when a measurement is carried out in the presence of magnetic particles 15.

In a third embodiment, illustrated in FIG. 10, the conductor 12 is integrated in the magnetoresistive sensor 11, thus forming an integrated sensor/conductor device 32. This integrated sensor/conductor device 32 both generates and detects the magnetic field. However, the allowable sensor current is now smaller than the conductor current I_{c} allowed in the previous embodiments due to power dissipation in the high ohmic sensor 32. Here again, a preliminary calibrating measurement is necessary.

Accuracy of (bio)sensors can be enhanced by knowing information about the concentration of magnetic particles as a function of position. By using any of the methods according to the present invention as described above, only the amount of magnetic particles 15 may be determined.

In a fourth embodiment, a device and method are described for determination of the concentration of magnetic material (e.g. nano beads) as a function of the location compared to the sensor 11.

A device according to this embodiment may comprise an integrated circuit having a magnetic sensor element 11, which may be, for example, a magnetoresistive sensor element such as e.g. a GMR or a TMR sensor element, and two conductors 12a-b, each at one side of he sensor element 11. A device according to this embodiment is illustrated in FIGS. 11 and 12 in perspective view and cross-section respectively.

FIG. 12 shows a cross sectional view of a device according to this embodiment. If the sensor device is positioned in the xy plane, the sensor 11 only detects a component of the magnetic field in a certain direction e.g. the x-component of a magnetic field, i.e. the x direction is the sensitive direction of the sensor 11. The sensitive direction is indicated by the arrow 13. Hence, magnetic fields 14a, 14b, caused by currents I₁ and I₂ flowing through the conductors 12a respectively 12b, will not be detected by the sensor 11 in absence of magnetic particles 15 as they are oriented in the z-direction at the location of the sensor 11.

In case magnetic particles, such as e.g. nano-particles 15, are present at the surface of the sensor 11, they each develop a magnetic moment m indicated by the field lines 16a, 16b in FIG. 12. The magnetic moments m generate dipolar stray fields which have in-plane magnetic field components 17a, 17b at the location of the sensor 11.

The z-component of the magnetic field H_{z} is roughly proportional to 1/x, or thus inversely proportional to the distance x between the magnetic particle 15 and the conductor. Therefore, the sensitivity of the detection mechanism depends on the position of the magnetic particle 15 at a particular position in the xy plane. More specifically, the responses of a magnetic particle 15 to currents I₁ and I₂ in the respective conductors 12a, 12b depend on the x-position of the magnetic particle 15 in the xy-plane, which can be seen from the graph in the lower part of FIG. 12. In this graph, the in-plane field strengths H_{x,1} and H_{x,2} induced by a magnetic particle 15 at position x in the xy plane in response to the conductor currents I₁ and I₂ is depicted.

By measuring H_{x,1} and H_{x,2} by time-, frequency- or phase (quadrature) multiplex techniques, the x-position of the magnetic particle 15 can be derived.

When the distance increases between the conductor (12a, 12b) and the sensor element (11), the magnetic field with respect to the surface plane of the magnetic sensor element (11) will become more perpendicular. This means that a magnetic nano-particle will become magnetized more perpendicularly. This results in a decrease in output response of the GMR sensor. The sensitivity of detection will therefore decrease more rapidly than 1/x, as mentioned here above.

The present invention includes within its scope sensors measuring more than one magnetic bead 15. In case a plurality of magnetic particles 15 are present, the sensor 11 measures an integral over the magnetic particle concentration as a function of the x-position of the sensor 11.

According to an embodiment, the magnetic particle concentration is determined as a function of the x-position by a frequency multiplex method, which is illustrated in FIG. 13. A first modulating signal Mod₁(t) is sent from a first source 20a to the first conductor 12a to modulate the current I₁ and is sent to a first demodulating multiplier 22a. The modulated current I₁ which flows through the conductor 12a induces a magnetic field, shown by field lines 14 in FIG. 12, which is mainly oriented perpendicular to the plane of the sensor element 11 at the location of the sensor 11. When magnetic particles 15 are present in the neighborhood of the sensor 11, the magnetic field at the location of the sensor 11 and thus the resistance of the sensor 11 is changed. The change of resistance gives rise to a different voltage drop over the sensor 11 and hence a different measurement signal delivered by the sensor 11. The measurement signal is sent through an amplifier 21 and the amplified measurement signal Ampl(t) is demodulated with the first modulating signal Mod₁(t). The resulting first intermediate signal Mult₁(t) is then sent through a first low pass filter 23a to form a first detection signal Det₁(t).

The current I₂ in the second conductor 12b is modulated by a second modulating signal Mod₂(t). The second modulating signal is sent to a second demodulating multiplier 22b where it is demodulated with the amplified measurement signal Ampl(t), thus forming a second intermediate signal Mult₂(t). The second intermediate signal Mult₂(t) is then sent through a second low pass filter 23b to form a second detection signal Det₂(t).

Both first and second detection signals Det₁(t) and Det₂(t) are applied to an interpreting means 34. These first and second detection signals Det₁(t) and Det₂(t) are a measure of the magnetic particles concentration in the sphere of influence of resp. I₁ and I₂. By interpreting these two detection signals Det₁(t), Det₂(t), information about the concentration distribution of the magnetic particles 15 may be retrieved.

A normalized difference signal PosX is given by:

$$PosX = \frac{{Det}_{1} ( t ) - {Det}_{2} ( t )}{{Det}_{1} ( t ) + {Det}_{2} ( t )}$$

and is representative for the average x-position of the magnetic particles 15.

The sum signal SUM=Det₁(t)+Det₂(t) is a measure for the total number of magnetic particles 15, their magnetization (diameter, permeability) and their position in a direction perpendicular to the plane of the sensor element 11, in the present case their z-position.

$$The ratio \text{:}$$

$$R = \frac{{Det}_{1} ( t )}{{Det}_{2} ( t )}$$

can also be used as an indication for the position of the magnetic particles 15 with respect to the sensitive direction of the sensor element 11, in the present case the x-position.

In case the frequency of Mod 1 and Mod 2 are the same, the magnetic field is zero in the middle of the sensor. By varying the amplitude balance of the two currents, the zero-point will shift along the x-axis. In this way additional information can be gathered about the particle distribution.

An advantage of the device described in the fourth embodiment above is that, in contrast to prior art techniques, the total chip area can be used for measurements. As a result hereof the chip area may be reduced with respect to the devices of the prior art. In FIG. 14 a cross-sectional view of a part of a sensor device according to the prior art of WO 03054523 is shown. The Fig. pictures only one half of a full Wheatstone bridge configuration used in the prior art. The sensor elements 35 are positioned next to each other at a distance of e.g. 3 μm. At the side opposite to the neighboring sensor element 35, 1.5 μm is left open. From the above it becomes clear that a 2*12 μm=24 μm strip width 36 is required to perform a single test. The bio-sensitive area 37, i.e. the working area of the device is 6 μm, as indicated in FIG. 14.

In the above described fourth embodiment of the present invention (FIG. 12) a bio-sensitive area 37 is achieved with a device a with strip width 36 of 6 μm (FIG. 15). A sensor element 11 is positioned in between two conductors 12a, 12b. If, for example, the sensor element 11 has a width of 3 μm as in the prior art device, and the distance between the edge of the sensor 11 and the middle of a conductor 12a, 12b is 1.5 μm, a total strip width of 6 μm is achieved. With respect to the prior art, the chip area may be reduced with a factor of 4, namely 2 times 12 μm versus 6 μm.

In a fifth embodiment of the present invention, an improved sensor device with respect to the previous embodiment is described. In order to distinguish between surface- and bulk concentrations of magnetic particles 15, resolution in a direction perpendicular to the plane of the sensor element 11, which corresponds to the z-direction with the co-ordinate system introduced in FIG. 16, is required. As shown in FIG. 16 conductors 12c and 12d generate a magnetic field 14c and 14d respectively in comparison with the magnetic field 14a and 14b of conductors 12a and 12b. By combining the sensor signals originating from the four conductors 12a, 12b, 12c, 12d, information may be obtained about the concentration of the magnetic particles 15 in x and z direction.

The z-resolution can be further enhanced by applying more conductors in the direction perpendicular to the plane of the sensor element 11, which as represented is the vertical or z direction. This is shown in the sixth embodiment in FIG. 17. Conductors 12a and 12b are positioned at both sides next to the magnetic sensor 11, at the same level in a direction perpendicular to the plane of the sensor element 11. Conductors 12c, 12d, 12e and 12f are positioned between the substrate 10 and the sensor 11, the conductors 12c and 12d are at a different z-position with respect to conductors 12e and 12f. Again, combination of the sensor signals resulting from the different conductors 12a to 12f may give information about the bulk and surface concentration of the magnetic particles 15.

In still another seventh embodiment, the currents in conductors 12c and 12d, which are positioned at a level in between the substrate 10 and the magnetic sensor 11, have opposite directions, as illustrated in FIG. 18. In that way, conductors 12c and 12d may generate a strong field gradient in the x direction. This embodiment may be advantageous for enhancing spatial resolution.

In embodiments 4 to 7 it is assumed that the position of a magnetic particle 15 does not change during the field scan measurement involving that magnetic particle 15. This assumption can be made because of the slow diffusion and the weak magnetic forces imposed by the current in the conductors 12a-12f.

The diffusion constant of a single magnetic bead, with a diameter of for example 100 nm, in an infinite volume of an aqueous solution at room temperature equals, according to the Stokes-Einstein formula, to:

$$D = \frac{kT}{6 πη R} = \frac{1.38 \cdot {10}^{- 23} \cdot 300}{6 π \cdot {10}^{- 3} \cdot 50 \cdot {10}^{- 9}} = 4.4 \cdot {10}^{- 12} {m}^{2} / s$$

From the formula a diffusion coefficient with a low value is achieved. When now applying for example a 10 MHz wobble frequency, the traveled distance of a magnetic particle 15 in one direction during 1 wobble period equals:

$$L = \sqrt{2 Dt} = \sqrt{2 \cdot 4.4 \cdot {10}^{- 12} \cdot {10}^{- 7}} = 1 nm$$

Assuming now 100 wobble periods per measurement, the displacement of the 100 nm nano-particles 15 equals 10 nm.

The magnetic force due to a magnetic field on a magnetic particle 15 can be encapsulated in a general formula:

$$F = ▽ ( mB ) \approx m ▽ B = m \frac{\partial B}{\partial w} m \frac{\partial ( \frac{{μ}_{0} I}{2 π w} )}{\partial w} = - m \frac{{μ}_{0} I}{2 π {w}^{2}}$$

If, for example, a 50 nm bead 15 is considered, and the magnetic moment m due to a current in the conductor 12 (I_{c}=20 mA) m≈6.10⁻¹⁴ Am², then for a sensor with GMR strip width w=3 μm, the magnetic attraction force equals:

$$F = 6 \cdot {10}^{- 18} \cdot \frac{4 π \cdot {10}^{- 7} \cdot 0.02}{2 π \cdot {( 3 \cdot {10}^{- 6} )}^{2}} = 2.7 fN$$

The velocity of a single particle 15 in an aqueous liquid as a result of the external force F equals:

$$v = \frac{F}{6 πη R} = \frac{2.7 \cdot {10}^{- 15}}{6 π \cdot {10}^{- 3} \cdot 50 \cdot {10}^{- 9}} = 2.9 μ m / s$$

In the situation where the particle 15 is actuated by the field of a single conductor 12 during 100 wobble periods, the displacement equals

$$x = v \cdot \frac{100}{f} = 2.9 \cdot {10}^{- 6} \cdot \frac{100}{{10}^{7}} = 30 pm$$

Therefore, this displacement may be neglected during performance of the measurements. The device and method described by the numerous embodiments of this invention have several advantages with respect to the prior art. First, the method has a small form factor. This means that:

(1) there is no alignment problem between generated magnetic field and sensor element, and

(2) only a low volume needs to be magnetized, which means that there is a low power consumption.

The biosensor itself and the interface circuitry can be small and low-power because of the absence of a coil, as it requires no external magnetic field.

Another advantage is the low power consumption due to the sensor being integrated. The device of the present invention has a power consumption of 10 mW versus 8 W in case of for example an external coil for driving the magnetic device as in the prior art. Furthermore, a high SNR is achieved due to 1/f noise removal and LF magnetic field suppression. Yet another advantage is that the detection method makes it possible to use sensor devices which require no surface structuring of the sensor device surface due to local field application. Nevertheless, surface patterning may be applied and will give additional benefits, such as e.g. no unnecessary loss of target molecules far away from the sensor.

Furthermore, a smaller chip area may be achieved, because 100% of the chip area may be used as bio-sensitive area or working area. Using the method according to the present invention, it is possible to make a distinction between surface and bulk concentration of magnetic particles 15 because of the spatial resolution in x and z direction. It is to be understood that although preferred embodiments, specific constructions and configurations, as well as materials, have been discussed herein for devices according to the present invention, various changes or modifications in form and detail may be made without departing from the scope and spirit of this invention.

For example, the present invention is not restricted to a single magnetoresistive sensor 11 but can also be applied in case of detection of magnetic particles 15 in multi-array biosensors. In that case a surrounding sensor element 1 may fulfill the functionality of conductor 12. This has the advantage that no extra conductor(s) 12 is/are necessary in a multi-assay bio-chip.

1. A magnetic sensor device for determining the presence of at least one magnetic particle (15), the magnetic sensor device comprising:
a magnetic sensor element (11) on a substrate (10),
a magnetic field generator (12) for generating an ac magnetic field,
a sensor circuit (3) comprising the magnetic sensor element (11) for sensing a magnetic property of the at least one magnetic particle (15) which magnetic property is related to the ac magnetic field, characterized in that the magnetic field generator (12) is integrated on the substrate (10) and is arranged to operate at a frequency of 100 Hz or above.

2. A magnetic sensor device as claimed in claim 1, characterized in that the magnetic field generator (12) is arranged to operate at a frequency where the thermal white noise of the magnetic sensor element (11) is dominant over the 1/f noise of the magnetic sensor element (11).

3. A magnetic sensor device as claimed in claim 1, characterized in that the sensor circuit (3) comprises an amplifier being connected to the magnetic sensor element (11), and the magnetic field generator (12) is arranged to operate at a frequency where the thermal white noise at the output of the amplifier (21) is dominant over the 1/f noise at the output of the amplifier (21).

4. A magnetic sensor device according to claim 1, wherein the magnetic field generator (12) comprises a conductor and an ac current source for generating an ac current flowing through the conductor.

5. A magnetic sensor device according to 4, wherein the direction (30) of the ac magnetic field is mainly perpendicular to the plane of the magnetic sensor element in the direct neighborhood of the magnetic sensor element.

6. A magnetic sensor device according to claim 1, wherein the magnetic field generator (12) and the sensor circuit (3) form an integrated circuit.

7. A magnetic sensor device according to claims 1, wherein said magnetic field generator (12) and said magnetic sensor element (11) are positioned adjacent each other above a substrate (10).

8. A magnetic sensor device according to claim 1, wherein said magnetic field generator (12) is positioned between said substrate (10) and said magnetic sensor element (11).

9. A magnetic sensor device according to claim 1, the magnetic sensor element (11) lying in a plane, wherein said magnetic field generator (12) is positioned adjacent one side of the magnetic sensor element (11) and a further magnetic field generator (12′) is positioned on the opposite side of the magnetic sensor element (11) at a same position with respect to a direction perpendicular (30) to the plane of the magnetic sensor element (11).

10. A magnetic sensor device according to claim 1, wherein said magnetic sensor element is a magnetoresistive sensor element.

11. A magnetic sensor device according to claim 1, furthermore comprising means for determining a concentration of magnetic particles.

12. A magnetic sensor device according to claim 11, wherein the means for determining a concentration of magnetic particles comprises a plurality of magnetic field generators.

13. A magnetic sensor device according to claim 12, the magnetic sensor element lying in a plane, wherein the plurality of magnetic field generators are located at different levels with respect to the plane of the magnetic sensor element.

14. A magnetic sensor device according to claim 1, wherein the at least one magnetic particle is a magnetic label coupled to a biological molecule.

15. A method for determining the presence of at least one magnetic particle (15), the method comprising the steps of:
generating an ac magnetic field in the vicinity of a magnetic sensor element (11),
sensing with the magnetic sensor element a magnetic property of the at least one magnetic particle (15) which magnetic property is related to the ac magnetic field, characterized in that the frequency of the ac magnetic field is chosen at 100 Hz or above.

16. A method as claimed in claim 15, characterized in that the frequency is chosen at a value where the thermal white (Nyquist) noise of the magnetic sensor element (11) is dominant over the 1/f noise of the magnetic sensor element (11).

17. A method as claimed in claim 15, characterized in that an amplifier (21) is connected to the magnetic sensor element (11) and the frequency of the ac magnetic field is chosen at a value where the thermal white noise at the output of the amplifier (21) is dominant over the 1/f noise at the output of the amplifier (21).

18. A method as claimed in claim 15, characterized in that the direction (30) of the generated ac magnetic field is mainly perpendicular to the plane of the magnetic sensor element in the direct neighborhood of the magnetic sensor element.

19. A method as claimed in claim 15, further comprising the steps of:
performing a calibrating measurement in absence of magnetic particles (15), which calibrating measurement measures the magnetic field generated by the magnetic field generator (12).
using the obtained calibrating measurement value and subtract that value from the actual measurement value obtained when a measurement is carried out in the presence of magnetic particles (15).

20. A method for determining a concentration of magnetic particles as a function of location of the magnetic particles by using the device of claim 9, wherein each of the magnetic field generators (12) generates an ac magnetic field with a different modulation (20a, 20b) frequency, the output signal of the magnetic sensor element (11) is demodulated resulting in signals with different frequency, from which signals the number of magnetic particles and the position is determined.

21. A method for determining the surface concentration and the bulk concentration of the magnetic particles by using the device of claim 13, wherein the plurality of magnetic field generators generate an ac magnetic field component normal (30) to the in-plane directions of the magnetic sensor element (11), from which magnetic field component the position of the magnetic particles is determined.

22. A method as claimed in claim 21, wherein each of the magnetic field generators generate an ac magnetic field with different modulation frequencies, the output signal of the magnetic sensor element is demodulated resulting in signals with different frequency, from which signals the number of magnetic particles and the position is determined.

23. Use of a method according to claim 15 for molecular diagnostics biological sample analysis, or chemical sample analysis.
